(19) United States
(12) Patent Application Publication
(10) Pub. No.: US 2015/0282828 A1
(43) Pub. Date: Oct. 8, 2015

(54) MEDICAL MANIPULATOR AND MEDICAL MANIPULATOR CONTROL METHOD

(71) Applicant: OLYMPUS CORPORATION, Tokyo (JP)

(72) Inventors: Kosuke KISHI, Tokyo (JP); Hiroshi WAKAI, Tokyo (JP)

(73) Assignee: OLYMPUS CORPORATION, Tokyo (JP)

(21) Appl. No.: 14/742,869

(22) Filed: Jun. 18, 2015

Related U.S. Application Data
(63) Continuation of application No. PCT/JP2014/054984, filed on Feb. 21, 2014.
(60) Provisional application No. 61/768,720, filed on Feb. 25, 2013.

(51) Int. Cl. A61B 17/29; A61B 1/045

(52) U.S. Cl. CPC A61B 17/2909; A61B 1/045; A61B 2017/00017

(57) ABSTRACT

A medical manipulator includes an inserting section which is configured to be inserted in a body, a treatment section that is provided in a distal end portion of the inserting section, an operating section that operates the inserting section, and a control section that includes a retraction mode in which the treatment section is retracted to a proximal end side when the control section receives an instruction to move the inserting section from the operating section.

This application is a continuation application based on PCT/JP2014/054984, filed on Feb. 21, 2014, claiming priority based on U.S. Patent Provisional Application No. 61/768,720, filed in United States on Feb. 25, 2013. The contents of both the United States Patent Provisional Application and the PCT Application are incorporated herein by reference.

### BACKGROUND OF THE INVENTION

1. Field of the Invention

The present invention relates to a medical manipulator that is used by being inserted in the body and a medical manipulator control method of controlling the medical manipulator.

2. Description of Related Art

In recent years, to save manpower in medical facilities, a medical treatment using a robot has been considered. In particular, in the field of surgery, various medical manipulator systems through which a patient is treated using a multi-degree of freedom (multijoint) manipulator have been proposed.

For example, in a surgical treatment apparatus disclosed in United States Patent Application, Publication No. 2008/0065109, a camera head of an imaging system is arranged at the distal end of a guiding tube (inserting section). A first work instrument and a second work instrument (treatment section) extend within a visual field of the imaging system from the distal end of the guiding tube. A volume allowed is determined to correspond with the boundary of the visual field. A control system of the surgical treatment apparatus prevents all parts of both the work instruments from moving beyond the allowed volume. Thus, since a surgeon can see all moving parts of the work instruments, the surgeon can move both the work instruments without touching surrounding tissues. The instrument volume is determined by the boundary where the work instruments are moving. The control system moves both the work instruments in a region of the allowed volume within the instrument volume.

When the imaging system is inserted toward a distal end side with respect to the guiding tube, the boundary of the visual field of the imaging system, that is, the volume allowed also moves to the distal end side, and a part of both the work instruments is placed outside the visual field of the imaging system.

### SUMMARY OF THE INVENTION

In order to solve the problem, the present invention proposes the following solutions.

According to a first aspect of the present invention, there is provided a medical manipulator including: an inserting section which is configured to be inserted in a body; a treatment section that is provided on a distal end portion of the inserting section; an operating section that operates the inserting section; and a control section that includes a retraction mode in which the treatment section is retracted to a proximal end side when the control section receives an instruction to move the inserting section from the operating section.

According to a second aspect of the present invention, in the medical manipulator according to the first aspect of the present invention, the control section may move the treatment section to the proximal end side only when the control section receives the instruction to move the inserting section in a direction intersecting an axis line of the inserting section from the operating section.

According to a third aspect of the present invention, in the medical manipulator according to the first or second aspect of the present invention, when the treatment section is moved to the proximal end side, the control section may move a distal end portion of the treatment section closer to the proximal end side than a position of the distal end portion of the treatment section when the control section receives the instruction.

According to a fourth aspect of the present invention, in the medical manipulator according to the third aspect of the present invention, the control section may store a position of an arrangement reference line of the treatment section defined along a longitudinal direction of the treatment section when the control section receives the instruction, and may move the treatment section to the proximal end side while maintaining a state where the treatment section is arranged on the arrangement reference line.

According to a fifth aspect of the present invention, in the medical manipulator according to any one of the first to third aspects of the present invention, when the control section receives the instruction, the control section may deform the treatment section into a linear shape extending to a distal end side thereof before the treatment section is moved to the proximal end side.

According to a sixth aspect of the present invention, in the medical manipulator according to the third aspect of the present invention, a channel which has an opening in a distal end surface of the inserting section may be formed in the inserting section, the treatment section may be inserted in the channel being capable of moving advanced and retracted, and the control section may move the treatment section so as to be housed in its entirety inside the channel when the control section receives the instruction.

According to a seventh aspect of the present invention, in the medical manipulator according to any one of the first to sixth aspects of the present invention, the control section may move the inserting section after a retraction operation of the treatment section is finished.

According to an eighth aspect of the present invention, in the medical manipulator according to any one of the first to sixth aspects of the present invention, the control section may move the inserting section after the instruction is received and before a retraction operation of the treatment section is finished.

According to a ninth aspect of the present invention, in the medical manipulator according to any one of the first to eighth aspects of the present invention, the control section may perform control so that the amount of movement of the treatment section toward the proximal end side is increased as the amount of movement of the inserting section indicated by the instruction is increased.

According to a tenth aspect of the present invention, in the medical manipulator according to any one of the first to ninth aspects of the present invention, the control section may have a non-retraction mode in which the treatment section is not moved to the proximal end side even though the instruction to move the inserting section is received from the operating section, and the control section may be capable of switching between the retraction mode and the non-retraction mode.

According to an eleventh aspect of the present invention, in the medical manipulator according to any one of the first to tenth aspects of the present invention, an imaging section which has an imaging unit capable of obtaining an image of a subject in a visual field range may be provided on the distal end portion of the inserting section, a distal end of the treatment section may be arranged in the visual field range of the imaging unit when the control section receives the instruction, the control section may store an initial visual field range that is the visual field range of the imaging unit when the instruction is received, may start the movement of the inserting section, and may stop the movement of the inserting section when the distal end of the treatment section reaches an edge part of the initial visual field range.

According to a twelfth aspect of the present invention, in the medical manipulator according to the seventh aspect of the present invention, the medical manipulator may further include an imaging section that includes an imaging section main body provided to protrude from a distal end surface of the inserting section and an imaging unit provided in a distal end portion of the imaging section main body and capable of obtaining an image of a subject, and the control section may stop the retraction operation of the treatment section when the treatment section moves to the proximal end side so that a distal end of the treatment section reaches the position of a distal end of the imaging section in a direction of an axis line of the inserting section.

According to a thirteenth aspect of the present invention, there is provided a medical manipulator control method of controlling a medical manipulator that includes an inserting section that is configured to be inserted in a body, a treatment section that is provided on a distal end portion of the inserting section, and an operating section that operates the inserting section, the method including: a process of moving the treatment section to a proximal end side when an instruction to move the inserting section is received from the operating section.

According to a fourteenth aspect of the present invention, in the medical manipulator control method according to the thirteenth aspect of the present invention, the treatment section may be moved to the proximal end side only when the instruction to move the inserting section in a direction intersecting an axis line of the inserting section is received from the operating section.

### BRIEF DESCRIPTION OF THE DRAWINGS

FIG. 1 is a general drawing illustrating a medical manipulator according to a first embodiment of the present invention.

FIG. 2 is a block diagram illustrating the medical manipulator.

FIG. 3 is a side view that a part of an endoscope inserting section of the medical manipulator is fractured.

FIG. 4 is a flowchart illustrating the procedure of a case where an operation instruction to change an angle of a bending section is output, in a control method of the medical manipulator.

FIG. 5 is a diagram illustrating the control method of the medical manipulator, which shows a distal end position of grasping forceps with time and a bending angle of the endoscope inserting section.

FIG. 6 is a diagram illustrating the control method of the medical manipulator.

FIG. 7 is a diagram illustrating the control method of the medical manipulator.

FIG. 8 is a diagram illustrating a control method of a medical manipulator according to a modification example of the first embodiment of the present invention.

FIG. 9 is a diagram illustrating the control method of the medical manipulator, which shows a distal end position of grasping forceps with time and a bending angle of an endoscope inserting section.

FIG. 10 is a side view that a part of an endoscope inserting section of a medical manipulator according to a second embodiment of the present invention is fractured.

FIG. 11 is a flowchart illustrating the procedure of a case where an operation instruction for changing an angle of a bending section is output, in a control method of the medical manipulator.

FIG. 12 is a diagram illustrating the control method of the medical manipulator.

FIG. 13 is a flowchart illustrating the procedure of a case where an operation instruction for changing an angle of a bending section is output, in a control method of a medical manipulator according to a third embodiment of the invention.

FIG. 14 is a diagram illustrating the control method of the medical manipulator.

FIG. 15 is a diagram illustrating the control method of the medical manipulator.

FIG. 16 is a side view that a part of an endoscope inserting section of a medical manipulator according to a fourth embodiment of the present invention is fractured.

FIG. 17 is a diagram illustrating an example of an image obtained by an imaging unit of the medical manipulator.

FIG. 18 is a diagram illustrating a control method of the medical manipulator, which shows a distal end position of grasping forceps with time and a bending angle of the endoscope inserting section.

FIG. 19 is a side view that a part of an endoscope inserting section of a medical manipulator according to a fifth embodiment of the present invention is fractured.

FIG. 20 is a block diagram illustrating the medical manipulator.

FIG. 21 is a diagram illustrating a control method of the medical manipulator.

FIG. 22 is a diagram illustrating the control method of the medical manipulator.

FIG. 23 is a diagram illustrating an operation of a medical manipulator according to an embodiment of a modification example of the present invention.

FIG. 24 is a diagram illustrating an operation of a medical manipulator according to an embodiment of a modification example of the present invention.

FIG. 25 is a diagram illustrating an example of the relationship between a movement amount of an operation arm and a movement amount to grasping forceps to a proximal end side according to an embodiment of a modification example of the present invention.

FIG. 26 is a diagram illustrating another example of the relationship between a movement amount of an operation arm and a movement amount to grasping forceps to a proximal end side according to an embodiment of a modification example of the present invention.

FIG. 27 is a diagram illustrating still another example of the relationship between a movement amount of an operation arm and a movement amount to grasping forceps to a proximal end side according to an embodiment of a modification example of the present invention.

### DETAILED DESCRIPTION OF THE INVENTION

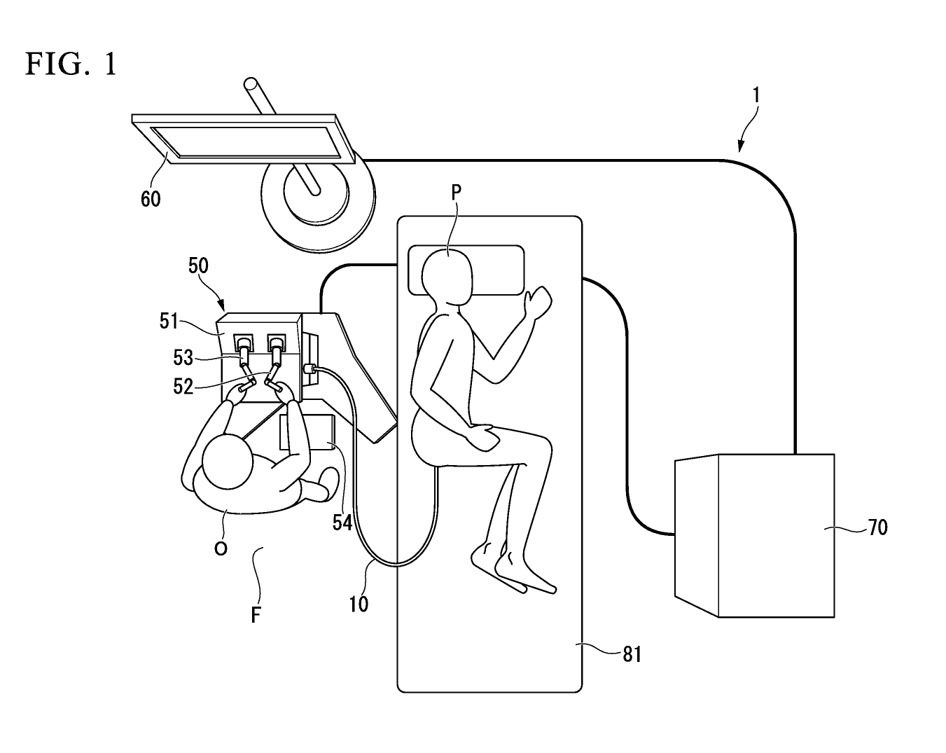

Hereinafter, a first embodiment of a medical manipulator 1 according to the present invention will be described with reference to FIGS. 1 to 9.

As shown in FIGS. 1 to 3, the medical manipulator 1 of the present embodiment includes an endoscope inserting section (inserting section) 10 inserted in the body, grasping forceps (treatment section) 30 that are retractably inserted in a channel 11 formed in the endoscope inserting section 10 being capable of moving advance and retracted, an operating section 50 that is operated by an operator O such as a practitioner to give an operation instruction (instruction), and a control device 70 that controls the endoscope inserting section 10 according to the operation instruction.

The endoscope inserting section 10 is a so-called flexible inserting section having flexibility. The endoscope inserting section 10 includes a rigid distal end portion 12 provided at the distal end thereof, a bending section 13 provided on the proximal end side of the rigid distal end portion 12 to be bendable, and a flexible tube part 14 that is provided on the proximal end side of the bending section 13 and that has flexibility.

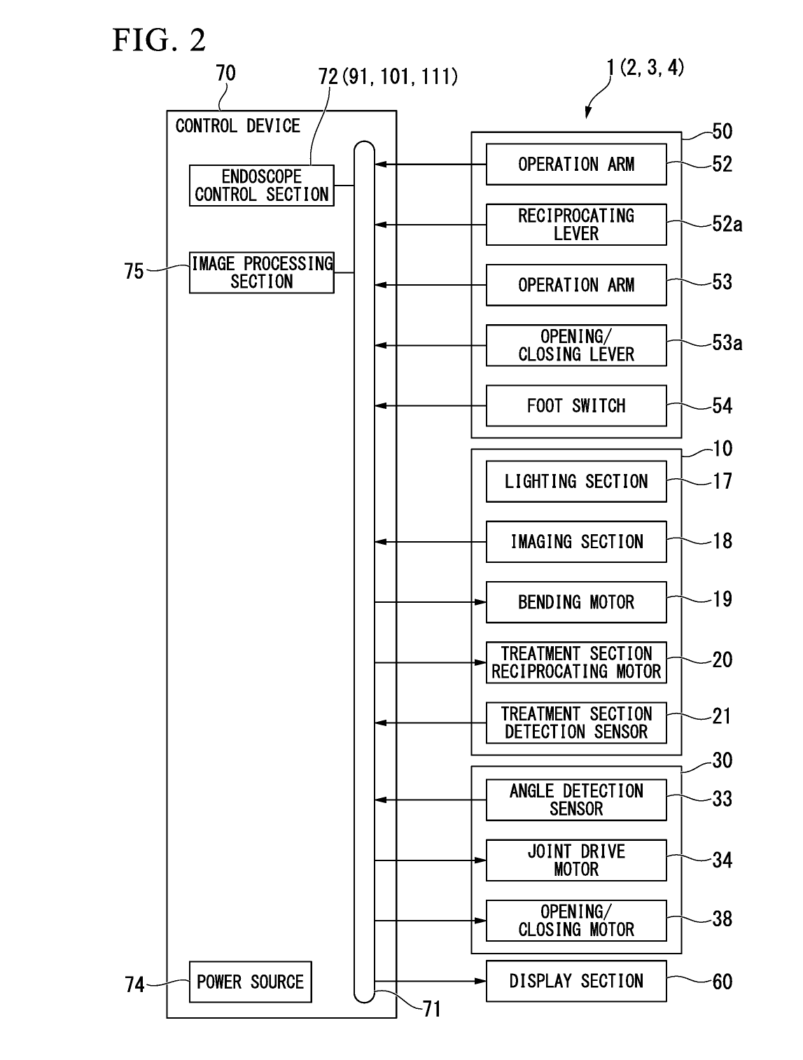

In a distal end surface 12a of the rigid distal end portion 12, a lighting section 17 (see FIG. 2) having an LED and an imaging section 18 having an imaging unit 18a such as a CCD are provided in a state of being exposed to the outside. The channel 11 has an opening 11a at the distal end surface 12a. The channel 11 is formed to extend in a direction of an axis line C1 of the endoscope inserting section 10.

The lighting section 17 is supplied with electric power from a power source 74 to be described later, and illuminates a front side of the distal end surface 12a. The imaging unit 18a obtains an image (picture) of the target tissue by imaging light reflected from target tissue (subject) in a predetermined viewing range R1 on a light receiving surface (not shown). Then, the image is converted into a signal and is output to the control device 70.

As the bending section 13, a bending section having a known configuration may be used. The bending section 13 is provided in a state where plural joint rings (not shown) that are rotatably connected to each other are arranged in the direction of the axis line C1. The joint ring of the distal end side among the plural joint rings is connected to distal end portions of four operating wires at equal angle intervals around the axis line C1. Proximal end portions of the respective operating wires are connected to a bending motor 19 (see FIG. 2) provided in a proximal end portion of the endoscope inserting section 10. By drawing the proximal end portion of the operating wire using the bending motor 19, the bending section 13 can be bent (moved) in a desired direction and thus, the angle of bending (bending angle) is changed.

In the proximal end portion of the endoscope inserting section 10, a treatment section reciprocating motor 20 shown in FIG. 2 used to advance and retract the grasping forceps 30 inserted in the channel 11 with respect to the channel 11 is provided. As a rotating shaft (not shown) of the treatment section reciprocating motor 20 rotates in a state of contacting an outer surface of the grasping forceps 30, it is possible to advance and retract the grasping forceps 30 with respect to the channel 11.

In the endoscope inserting section 10, a treatment section detection sensor 21 that detects the position, in the direction of the axis line C1, of the grasping forceps 30 inserted in the channel 11.

The proximal end portion of the endoscope inserting section 10 is attached to the operating section 50.

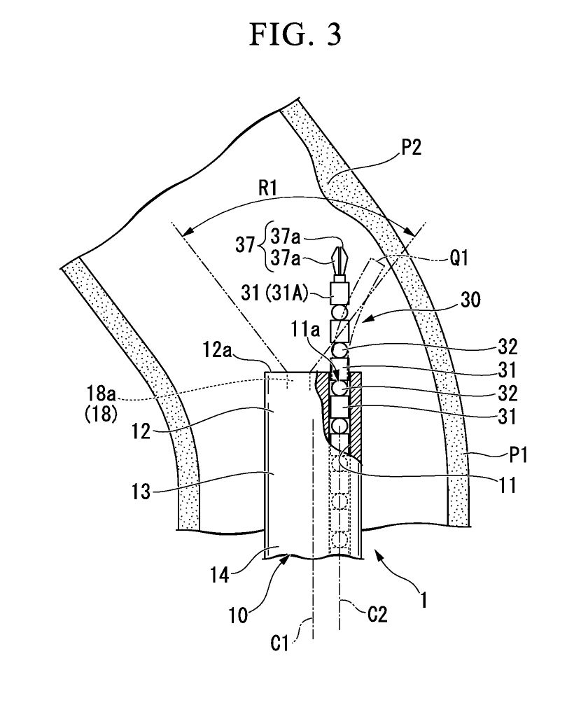

In the present embodiment, as the grasping forceps 30, as shown in FIG. 3, a so-called multijoint rod-shaped treatment instrument that extends in the longitudinal direction is used. The grasping forceps 30 includes plural tubular bodies 31 that are arranged in the direction of an axis line C2 of the grasping forceps 30, and has a configuration in which the tubular bodies 31 adjacent to each other in the direction of the axis line C2 are connected by a joint part 32. That is, a portion of the grasping forceps 30 where the tubular body 31 is provided is rigid and a portion of the grasping forceps 30 where the joint portion 32 is provided is rotatable. In addition, a part of the grasping forceps 30 is a rigid treatment section.

In the joint part 32, as shown in FIG. 2, an angle detection sensor 33 for detecting an angle formed by the adjacent tubular bodies 31 such as an encoder or a potentiometer and a joint drive motor 34 used to adjust the angle are provided.

By driving the respective joint drive motors 34, it is possible to bend the grasping forceps 30 in various shapes.

Each angle detection sensor 33 converts the detected angle into a signal and outputs the signal to the control device 70. The joint drive motor 34 is driven by the control device 70.

As shown in FIG. 3, a grasping section 37 having a pair of grasping pieces 37a is fixed to a tubular body 31A arranged on the extreme distal end side among the plural tubular bodies 31. A distal end portion of the operating wire (not shown) is connected to a proximal end portion of each grasping piece 37a. The operating wire is inserted into each tubular body 31 or the joint part 32, and is extended to the proximal end side of the grasping forceps 30. The proximal end portion of the operating wire is connected to an opening/closing motor 38 (see FIG. 2) provided in the proximal end portion of the grasping forceps 30. By retracting or advancing the proximal end portion of the operating wire by the opening/closing motor 38, it is possible to perform an opening/closing operation that the distal end sides of the pair of grasping pieces 37a separates from or close to each other.

As shown in FIGS. 1 and 2, the operating section 50 includes a pair of operation arms 52 and 53 attached to an operating board 51, and a foot switch 54 arranged on a floor surface F.

The operation arms 52 and 53 have a multijoint structure, and operate to bend the bending section 13 of the endoscope inserting section 10 and the grasping forceps 30. In the present embodiment, the amount of movement of the grasping forceps 30 toward the proximal end side when the operation arm 52 is operated is constant, which will be described next. That is, regardless of the amount of movement of the distal end portion of the operation arm 52 operated by the operator O, the amount of movement of the grasping forceps 30 toward the proximal end side is constant.

In the distal end portion of the operation arm 52, a reciprocating lever 52a (see FIG. 2) used to advance and retract the grasping forceps 30 with respect to the channel 11 by driving the treatment section reciprocating motor 20 is provided. In the distal end portion of the operation arm 53, an opening/closing lever 53a used to open and close the pair of grasping pieces 37a is provided.

The foot switch 54 performs switching of a control mode of an endoscope control section 72, as described later.

The operation arms 52 and 53, the reciprocating lever 52a, the opening/closing lever 53a and the foot switch 54 output operation instructions to the control device 70 when operated.

As shown in FIG. 1, at a position facing the operator O who holds the operation arms 52 and 53 using the hands, a display section 60 for displaying an image or the like obtained by the imaging unit 18a of the endoscope inserting section 10 is provided. The display section 60 is connected to the control device 70.

As shown in FIG. 2, the control device 70 includes the endoscope control section (control section) 72 connected to a bus 71, an image processing section 73, and a power source 74.

The imaging section 18, the bending motor 19, the treatment section reciprocating motor 20 and the treatment section detection sensor 21 of the endoscope inserting section 10, the angle detection sensor 33, the joint drive motor 34 and the opening/closing motor 38 of the grasping forceps 30, the operation arms 52 and 53, the reciprocating lever 52a, the opening/closing lever 53a and the foot switch 54 of the operating section 50, and the display section 60 are each connected to the bus 71.

The endoscope control section 72 and the image processing section 73 are configured of an arithmetic element, a memory, a control program and the like.

The endoscope control section 72 drives the bending motor 19 according to an operation instruction, which is outputted from the operation arm 52, with respect to the bending section 13 of the endoscope inserting section 10 to draw an appropriate operating wire and bend the bending section 13. The endoscope control section 72 drives each joint drive motor 34 according to an operation instruction, which is outputted from the operation arm 53, with respect to the grasping forceps 30 and bend the grasping forceps 30.

In the memory of the endoscope control section 72, the length of the tubular body 31 and the grasping section 37 or the like is stored. The arithmetic element of the endoscope control section 72 can calculate the shape of the grasping forceps 30, the position of the grasping section 37 of the grasping forceps 30 with respect to the distal end surface 12a of the endoscope inserting section 10, or the like based on the position of the grasping forceps 30 with respect to the channel 11 in the direction of the axis line C1, the angle detected by the angle detection sensor 33, and respective values stored in the memory. Here, the shape of the grasping forceps 30 includes a linear shape, a bent shape, or the like.

The endoscope control section 72 has two control modes. Specifically, one control mode is a retraction mode in which the grasping forceps 30 is automatically retracted to the proximal end side when an operation instruction to bend the bending section 13 is received. Another control mode is a non-retraction mode in which the grasping forceps 30 are not automatically moved to the proximal end side even though the operation instruction is received.

The endoscope control section 72 can switch the control modes between the retraction mode and the non-retraction mode according to the operation instruction from the foot switch 54.

The image processing section 73 appropriately converts the image signal which is outputted from the imaging unit 18a and outputs the result to the display section 60.

The power source 74 supplies electric power which is inputted from the outside to the endoscope inserting section 10, the grasping forceps 30, the operating section 50, the endoscope control section 72 or the like.

Next, a technique of using the medical manipulator 1 of the present embodiment configured as described above will be described. Hereinafter, a control method of the medical manipulator that changes the angle of the bending section 13 of the endoscope inserting section 10 when the grasping forceps 30 protrudes from the distal end surface 12a of the endoscope inserting section 10 will be mainly described. Hereinafter, a case of treating a target tissue formed in the wall of the large intestine will be described, but a target portion is not limited thereto, and for example, hollow organs such as an esophagus, stomach, duodenum, small intestine, uterus or bladder may be used.

As shown in FIG. 1, a care giver (not shown) puts a patient P to bed on an surgical table 81 and performs an appropriate treatment such as disinfecting or anesthesia. The surgical table 81 is placed beside the operating section 50. When the medical manipulator 1 is started up, electric power is supplied to the endoscope inserting section 10, the grasping forceps 30, the operating section 50, the endoscope control section 72 or the like from the power source 74.

As the electric power is supplied to the lighting section 17 from the power source 74, the front side of the endoscope inserting section 10 is illuminated. The operator O confirms an image of the front side of the endoscope inserting section 10 obtained by the imaging unit 18a through the display section 60 while grasping the operation arms 52 and 53.

The operator O operates the reciprocating lever 52a so as not to protrude the grasping forceps 30 does not protrude from the distal end portion of the channel 11. The operator O sets the control mode of the endoscope control section 72 into the retraction mode by operating the foot switch 54.

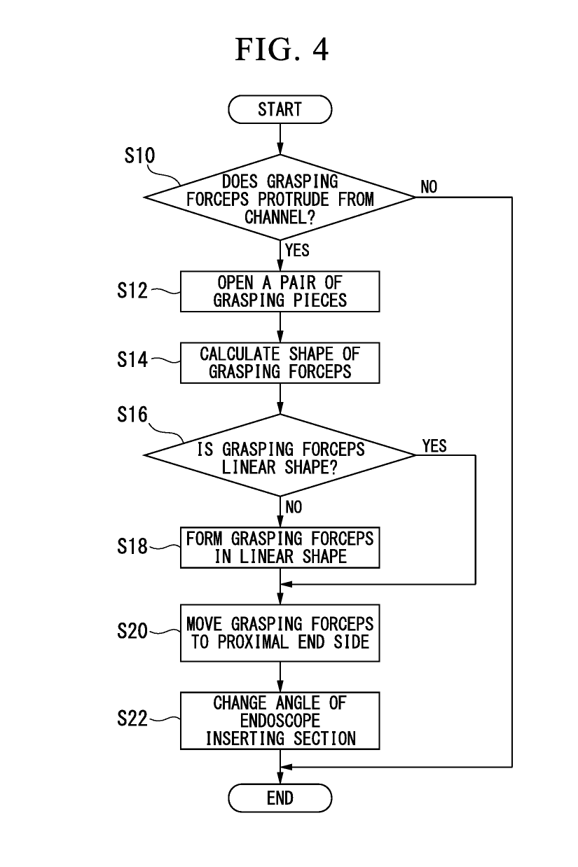

The operator O instructs the care giver to introduce the endoscope inserting section 10 into the large intestine P1 through the anus of the patient P, as shown in FIG. 3. The operator O operates the operation arm 52 to output an operation instruction with respect to the bending section 13 of the endoscope inserting section 10, thereby appropriately changing the angle of the bending section 13. Thus, the operator O causes the distal end surface 12a of the endoscope inserting section 10 to face target tissue P2 formed in the wall of the large intestine P1. By changing the angle of the bending section 13, the flow for an angle change instruction shown in FIG. 4 is performed. That is, in step S10, it is determined whether the grasping forceps 30 protrudes from the distal end portion of the channel 11. Since the grasping forceps 30 does not protrude from the channel 11 (NO), the flow for the angle change instruction ends.

Here, the operator O instructs the care giver to stop the introduction of the endoscope inserting section 10 into the large intestine P1 in a state where the endoscope inserting section 10 faces the target tissue P2.

The operator O operates the reciprocating lever 52a to cause the grasping forceps 30 to protrude from the distal end portion of the channel 11. Here, by operating the operation arm 53, the grasping forceps 30 is bent appropriately. The operator O operates the opening/closing lever 53a to treat the target tissue P2 appropriately. Hereinafter, a case where the grasping forceps 30 is placed in linear shape, that is, the grasping forceps 30 is placed along the channel 11 as shown in FIG. 3 after the treatment of the target tissue P2 will be described.

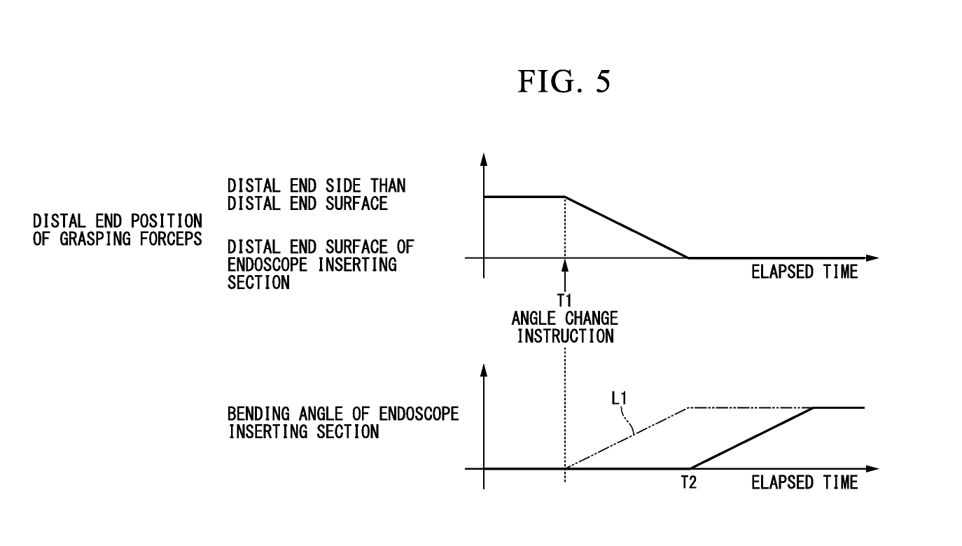

In order to observe the periphery of the target tissue P2, the operator O changes the angle of the bending section 13. Specifically, if the bending angle of the bending section 13 is increased, the flow for the angle change instruction shown in FIG. 4 is performed. As shown in FIG. 5, at time T1 when an operation instruction to change the angle is outputted, the grasping forceps 30 protrudes from the channel 11.

The flow shown in FIG. 4, first, in step S10, it is determined whether the grasping forceps 30 protrudes from the distal end portion of the channel 11. In this case, since the grasping forceps 30 protrudes from the channel 11 (YES), the procedure goes to step S12.

In step S12, the arithmetic element of the endoscope control section 72 drives the opening/closing motor 38 to open the pair of grasping pieces 37a, and then the procedure goes to step S14. This is performed to release the tissue from the grasping section 37 in a case where the tissue or the like is grasped between the pair of grasping pieces 37a.

In step S14, as described above, the shape of the grasping forceps 30 is calculated based on the angle and the like detected by the angle detection sensor 33, and then the procedure goes to step S16.

In step S16, it is determined whether the shape of the grasping forceps 30 is in the linear shape. In this case, since the grasping forceps 30 is in the linear shape (YES), the procedure goes to step S20.

In step S20, by operating the treatment section reciprocating motor 20 maintaining the grasping forceps 30 in the linear shape, the grasping forceps 30 is moved to the proximal end side. That is, the grasping forceps 30 is moved to the proximal end side so as not to drive the joint drive motor 34 of the grasping forceps 30 and not to change the angle formed by the adjacent tubular bodies 31. Accordingly, as shown in FIG. 5, the distal end portion of the grasping forceps 30 moves to the proximal end side with the lapse of time. When the grasping forceps 30 is moved to the proximal end side, the movement is performed maintaining the pair of grasping pieces 37a being closed.

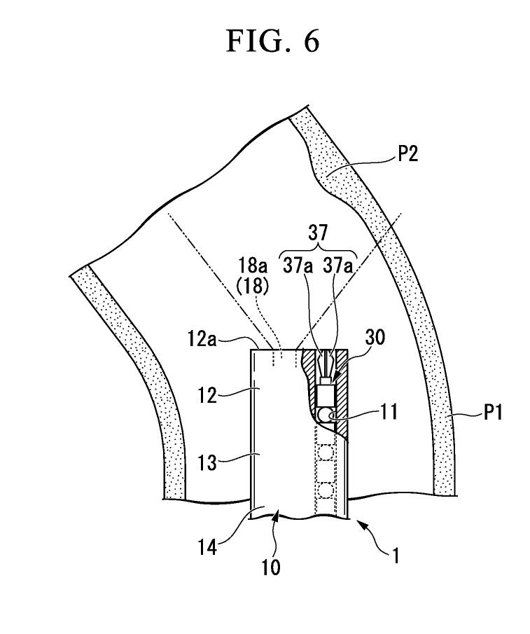

Further, as shown in FIGS. 5 and 6, the entirety of the grasping forceps 30 is housed inside the channel 11. In other words, the grasping forceps 30 is moved closer to the proximal end side than the distal end surface 12a of the endoscope inserting section 10. Accordingly, the retraction operation of the grasping forceps 30 used to house the entirety of the grasping forceps 30 in the channel 11 ends.

At the time of T2 when the entirety of the grasping forceps 30 is housed inside the channel 11, the procedure goes to step S22.

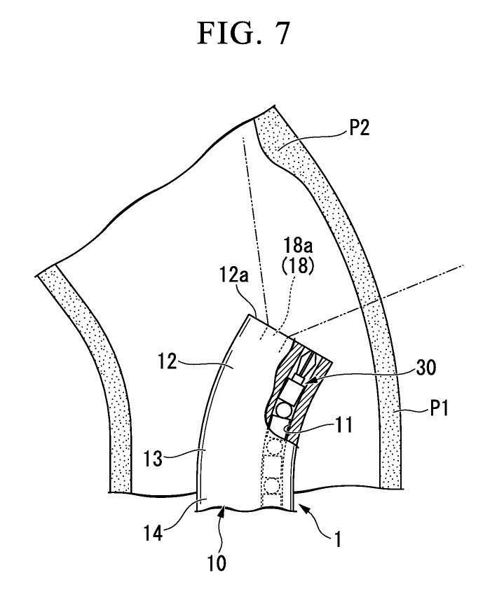

In step S22, according to the operation instruction for changing the angle, as shown in FIGS. 5 and 7, the bending angle of the bending section 13 is increased to bend the endoscope inserting section 10. By moving the grasping forceps 30 to the proximal end side, when the angle of the bending section 13 of the endoscope inserting section 10 is changed, a swiveling range of the grasping forceps 30 is narrowed. Further, since the entirety of the grasping forceps 30 is inside the channel 11, even though the angle of the bending section 13 of the endoscope inserting section 10 is changed, the grasping forceps 30 does not come into contact with the wall of the large intestine P1 or the like.

As indicated by a virtual line L1 in FIG. 5, the bending section 13 starts to be bent at the time of T2 when a predetermined time elapses from the time T1, not at the time of T1 when the operation instruction is outputted.

In the present embodiment, only after the angle of the bending section 13 of the endoscope inserting section 10 is changed, that is, only after receiving the operation instruction for moving the rigid distal end portion 12 of the endoscope inserting section 10 in a direction orthogonal to (intersecting) the axis line C1, the grasping forceps 30 may be moved to the proximal end side.

On the other hand, in a case where the grasping forceps 30 is bent as indicated by position Q1 in FIG. 3 at time T1 when the operation instruction for changing the angle is outputted, it is determined as NO in step S16, and then the procedure goes to step S18.

In step S18, the joint drive motor 34 is driven to deform the grasping forceps 30 in the linear shape extending to the distal end side, and then the procedure goes to step S20. The procedure of step S20 and thereafter is the same as the above-described procedure, and the description thereof will not be repeated here.

In a case of being protruded the grasping forceps 30 from the distal end portion of the channel 11 again, the operator O operates the reciprocating lever 52a to drive the treatment section reciprocating motor 20, thereby moving the grasping forceps 30 to the distal end side.

In a case that the endoscope inserting section 10 is moved slightly in a state where the grasping forceps 30 protrudes from the distal end portion of the channel 11, the operator O operates the foot switch 54 to switch the control mode of the endoscope control section 72 to the non-retraction mode.

If the treatment for the target tissue P2 is finished, the operator O instructs the care giver to take the endoscope inserting section 10 out the large intestine P1. Then, the operator O performs necessary treatments and completes the series of techniques.

According to the medical manipulator 1 and the control method of the medical manipulator 1 in the present embodiment, the endoscope control section 72 moves the grasping forceps 30 to the proximal end side when the operation instruction to bend the endoscope inserting section 10 is received. Thus, when the distal end side of the endoscope inserting section 10 is bent, the swiveling range of the grasping forceps 30 becomes narrowed. Accordingly, it is possible to suppress the grasping forceps 30 from contacting the wall of the large intestine P1 or the like.

According to the medical manipulator 1 and the control method of the medical manipulator 1 in the present embodiment, the endoscope control section 72 may move the grasping forceps 30 to the proximal end side only when the operation instruction to move the rigid distal end portion 12 of the endoscope inserting section 10 to the direction orthogonal to (intersecting) the axis line C1 is received. When the endoscope inserting section 10 moves in the direction orthogonal to the axis line C1, the grasping forceps 30 is likely to come into contact with a tissue or the like in a radial direction of the endoscope inserting section 10 with respect to the endoscope inserting section 10. Accordingly, it is possible to effectively suppress the endoscope inserting section 10 from contacting surrounding tissue of the large intestine P1 or the like.

In the medical manipulator 1 of the present embodiment, the endoscope control section 72 may house the entirety of the grasping forceps 30 inside the channel 11 when the operation instruction for changing the angle of the bending section 13 is received. Thus, it is possible to reliably prevent the grasping forceps 30 from contacting the wall of the large intestine P1 or the like.

In the medical manipulator 1 of the present embodiment, the endoscope control section 72 may bend the endoscope inserting section 10 after the retraction operation of the grasping forceps 30 is finished. Accordingly, it is possible to more reliably prevent the grasping forceps 30 from contacting the wall of the large intestine P1 or the like.

In the medical manipulator 1 of the present embodiment, it may be possible to switch the control modes between the retraction mode and the non-retraction mode by the endoscope control section 72. In this case, the control mode is switched to the non-retraction mode when the endoscope inserting section 10 is to be moved slightly and is switched to the retraction mode when the endoscope inserting section 10 is to be large. Thus, it is possible to adjust the control mode according to the operation content of the endoscope inserting section 10 or the surrounding situations.

In the medical manipulator 1 of the present embodiment, when the operation instruction to change the angle is received and when it is detected that the grasping forceps 30 is bent, the endoscope control section 72 may move the grasping forceps 30 to the proximal end side after deforming the grasping forceps 30 in the linear shape extending to the distal end side. Thus, it is possible to easily house the grasping forceps 30 in the channel 11 and to suppress the grasping forceps 30 from being damaged when housing the entirety of the grasping forceps 30 inside the channel 11.

In this case, only in the case of being protruded when the grasping forceps 30 from the channel 11, step S12 to step S22 are performed. Thus, it is possible to reduce burden necessary for controlling by the arithmetic element of the endoscope control section 72, and to effectively perform the control by the endoscope control section 72.

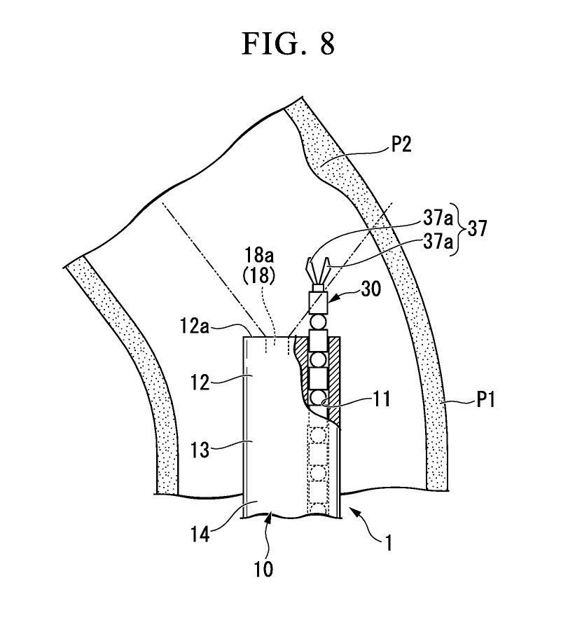

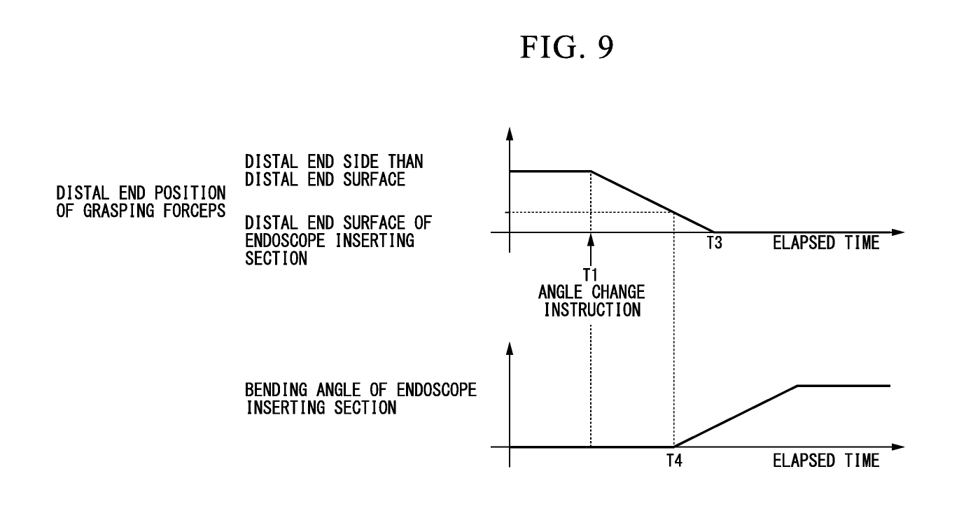

In the medical manipulator 1 of the present embodiment, the endoscope control section 72 may start the bending of the grasping forceps 30 before finishing the retraction operation of the grasping forceps 30 and after receiving the operation instruction to bend the bending section 13. More specifically, the grasping forceps 30 protruded from the channel 11 starts the bending of the endoscope inserting section 10 at the time of T4 prior to the time of T3 when the entirety of the grasping forceps 30 is housed inside the channel 11, as shown in FIGS. 8 and 9.

According to this control by the endoscope control section 72, it is possible to shorten the interval from the time of receiving the operation instruction to bend the bending section 13 to the time of actually starting to bend of the grasping forceps 30. Thus, when the operator O operates the operating section 50, it is possible to reduce the operator O discomfort of the endoscope inserting section 10 seldom bending even while the operator O operates the operating section 50.

In the medical manipulator 1 of the present embodiment, the endoscope inserting section 10 may be connected to an arm portion of a multijoint robot. The multijoint robot may be connected to the endoscope control section 72 through the bus 71. If the operator O operates the operation arm 52 of the operating section 50, for example, an operation instruction to move the endoscope inserting section 10 to the distal end side is outputted from the operating section 50. Here, the endoscope control section 72 operates the arm portion of the multijoint robot to move the endoscope inserting section 10 to the distal end side. On the other hand, the endoscope control section 72 moves the grasping forceps 30 to the proximal end side with respect to the channel 11.

By controlling the endoscope control section 72 as described above, it is also possible to suppress the grasping forceps 30 from contacting the wall of the large intestine P1 or the like.

### Second Embodiment

Next, a second embodiment of the invention will be described with reference to FIGS. 2, 10 to 12, in which the same reference numerals are given to the same parts as in the first embodiment and a description thereof will not be repeated here.

As shown in FIG. 2, a medical manipulator 2 of the present embodiment includes an endoscope control section 91 instead of the endoscope control section 72 of the medical manipulator 1 in the first embodiment. That is, the medical manipulator 2 is different from the medical manipulator 1 only in the control content of the endoscope inserting section 10 and the grasping forceps 30.

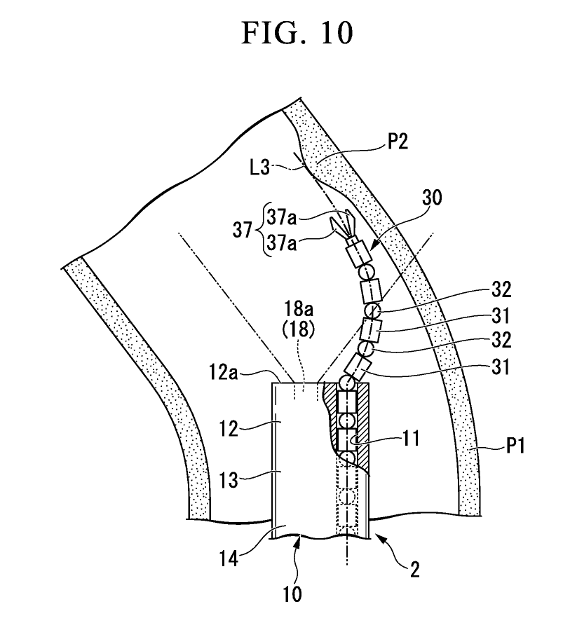

The endoscope control section 91 calculates the position of an arrangement reference line L3 of the grasping forceps 30 defined along the longitudinal direction of the grasping forceps 30 shown in FIG. 10 when the operation instruction to change the angle is received, and stores the position of the calculated arrangement reference line L3. More specifically, the endoscope control section 91 can calculate the shape of the grasping forceps 30 as described above. The endoscope control section 91 specifies a central axis line of the grasping forceps 30 when the operation instruction is received, for example, as the arrangement reference line L3, and stores the position of the arrangement reference line L3 for a reference coordinate system defined with respect to the surgical table 81 in the memory of the endoscope control section 91, for example.

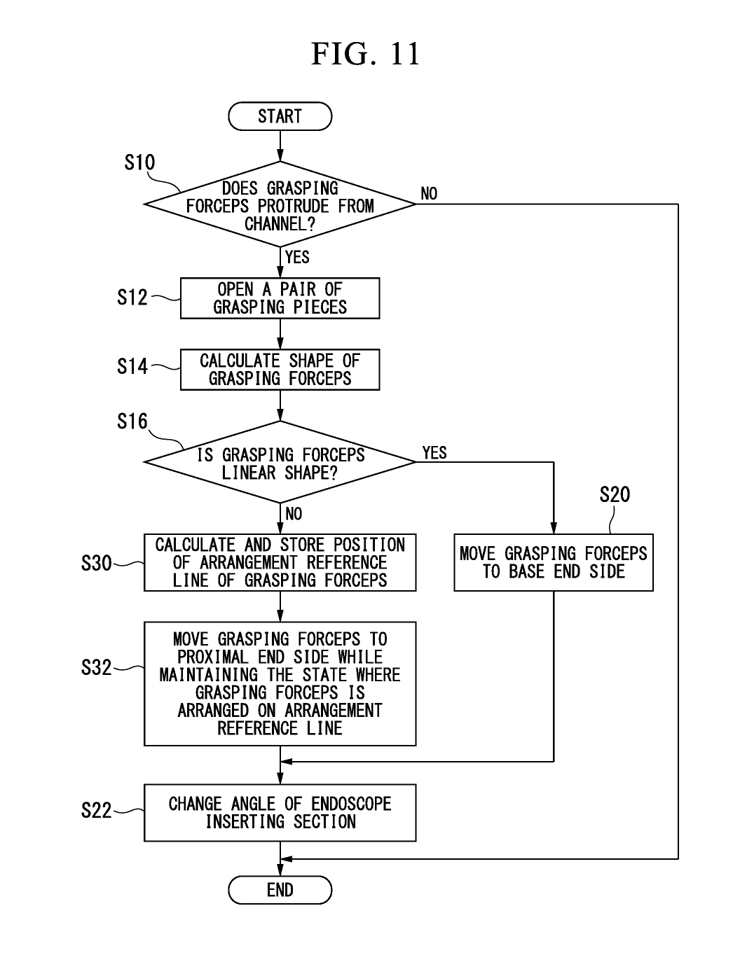

A technique of using the medical manipulator 2 of the present embodiment with such a configuration will be described in a point different from the technique of using the medical manipulator 1 of the first embodiment. FIG. 11 is a flowchart illustrating the procedure when the operation instruction for changing the angle of the bending section 13 is outputted. Hereinafter, a case where the grasping forceps 30 is bent as shown in FIG. 10 after treatment of the target tissue P2 will be described.

In step S16, it is determined whether the shape of the grasping forceps 30 is in the linear shape. Since the grasping forceps 30 is bent (NO), the procedure goes to step S30.

In step S30, the endoscope control section 91 calculates the position of the arrangement reference line L3 of the grasping forceps 30 as described above, and stores the position of the calculated arrangement reference line L3 in the memory. Then, the procedure goes to step S32.

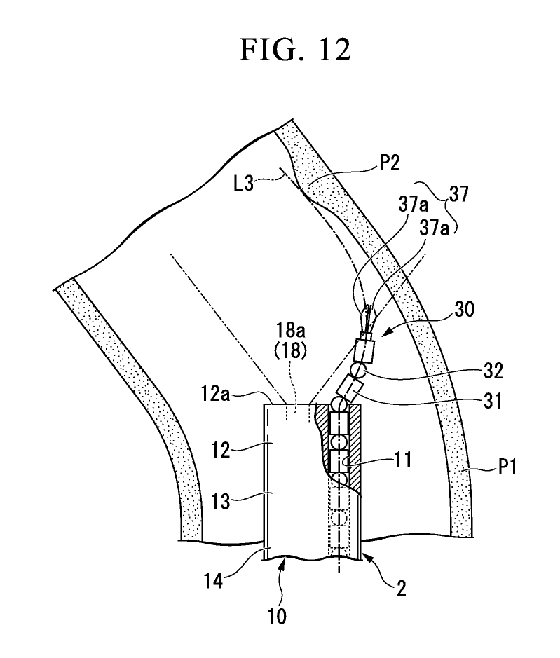

In step S32, the endoscope control section 91 drives each joint drive motor 34 and the treatment section reciprocating motor 20, and moves the grasping forceps 30 to the proximal end side while maintaining the state where the grasping forceps 30 is arranged on the arrangement reference line L3, as shown in FIG. 12. As a method of moving the grasping forceps 30 while maintaining the state where the grasping forceps 30 is arranged on the arrangement reference line L3, for example, a known method such as a curve propagation method disclosed in U.S. Pat. No. 6,468,203 may be used.

Briefly describing this method, the endoscope inserting section is configured so that sections thereof are arranged in an axial direction. The adjacent sections are rotatable by a linear actuator. When the endoscope inserting section is retracted from a bent pipe, the bending angle between the adjacent sections is set to a bending angle between the adjacent sections on the proximal end side corresponding to one section, for each retraction of the endoscope inserting section corresponding to one section. By controlling the bending angle between sections in this wise, the endoscope inserting section is seen as being fixed in a space.

According to the number of the joint parts 32 of the grasping forceps 30, and to the length of the tubular body 31 or the like, it is considered that the grasping forceps 30 may not be maintained in the state of being arranged on the arrangement reference line L3 when the grasping forceps 30 is moved to the proximal end side. Even in such case, the endoscope control section 91 controls the grasping forceps 30 to be arranged on the arrangement reference line L3 as much as possible.

If the grasping forceps 30 is moved to the proximal end side to be housed the entirety of the grasping forceps 30 in the channel 11, the procedure goes to step S22.

Since the procedure of step S22 and thereafter is the same as in the first embodiment, a description thereof will not be repeated here.

On the other hand, when the grasping forceps 30 is in the linear shape after the treatment of the target tissue P2, the determination result in step S16 is YES, and then the procedure goes to step S20. Since the procedure of step S20 and thereafter is the same as in the first embodiment, a description thereof will not be repeated here.

According to the medical manipulator 2 of the present embodiment with such a configuration, while the grasping forceps 30 is moved to the proximal end side, since the grasping forceps 30 are not likely to come into contact with tissue other than the tissue being in contact therewith when the operation instruction is received, it is possible to suppress the grasping forceps 30 from contacting the wall of the large intestine P1 or the like.

If the bent grasping forceps 30 extends to be in the linear shape, there is a possibility that the distal end portion of the grasping forceps 30 comes close to and into contact with tissue on the front side. However, by moving the grasping forceps 30 to the proximal end side along the arrangement reference line L3, it is possible to help prevent the distal end portion of the grasping forceps 30 from contacting the tissue.

### Third Embodiment

Next, a third embodiment of the present invention will be described with reference to FIGS. 2, 13 to 15, in which the same reference numerals are given to the same parts as in the embodiments, and a description thereof will not be repeated here.

As shown in FIG. 2, a medical manipulator 3 of the present embodiment includes an endoscope control section 101 instead of the endoscope control section 72 of the medical manipulator 1 in the first embodiment.

When moving the grasping forceps 30 to the proximal end side, the endoscope control section 101 moves the distal end portion of the grasping forceps 30 to the proximal end side without moving the grasping forceps 30 closer to the distal end side than the position at the time of receiving the operation instruction to change the angle.

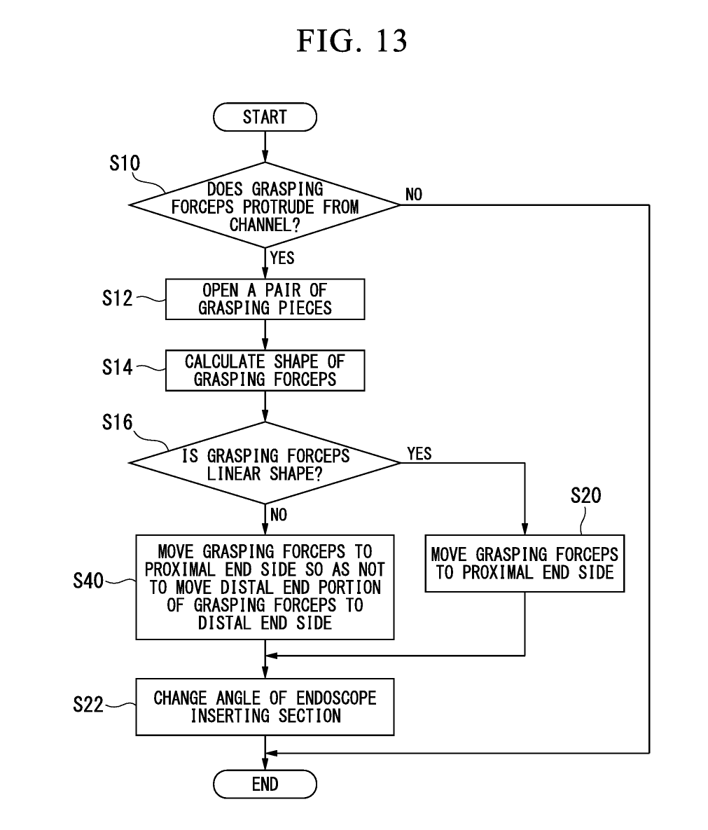

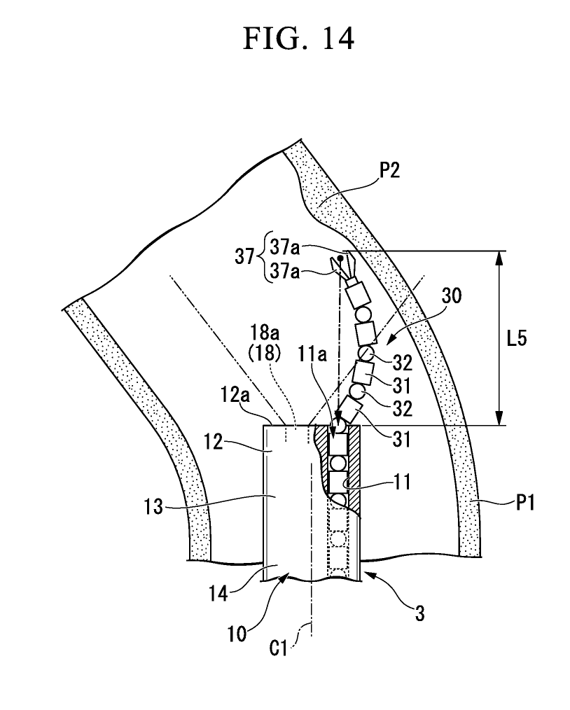

A technique of using the medical manipulator 3 of the present embodiment will be described in a point different from the technique of using the medical manipulator 1 of the first embodiment. FIG. 13 is a flowchart illustrating the procedure in a case of outputting the operation instruction to change the angle of the bending section 13. Hereinafter, it is assumed that the grasping forceps 30 is bent as shown in FIG. 14 after treatment of the target tissue P2. Here, the length along the axis line C1 from the distal end surface 12a of the endoscope inserting section 10 to the distal end of the grasping forceps 30 is represented as L5.

In step S16 in FIG. 13, it is determined whether the shape of the grasping forceps 30 is in the linear shape. Since the grasping forceps 30 is bent (NO), the procedure goes to step S40.

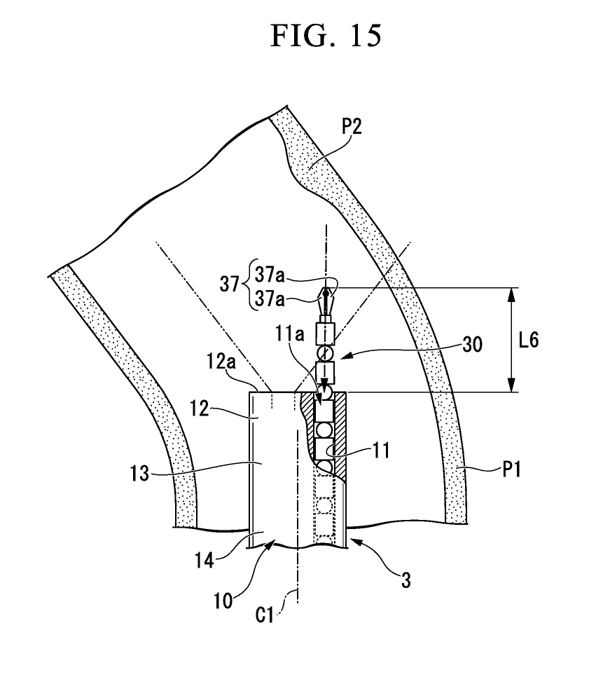

In step S40, the endoscope control section 101 moves the grasping forceps 30 to the proximal end side. Here, as shown in FIG. 15, the distal end portion of the grasping forceps 30 is moved to the proximal end side without moving the distal end portion of the grasping forceps 30 closer to the distal end side than the position at the time of receiving the operation instruction to change the angle. Further, the distal end portion of the grasping forceps 30 is moved linearly to the opening 11a of the channel 11. When the distal end portion of the grasping forceps 30 is moved to the proximal end side, the length along the axis line C1 from the distal end surface 12a of the endoscope inserting section 10 to the distal end of the grasping forceps 30 is represented as L6 which is shorter than the above-mentioned length L5. As a method of controlling parts other than the distal end portion in the grasping forceps 30, a desired control method such as a known compliance control may be used.

Then, the procedure goes to step S22. Since the procedure of step S22 and thereafter is the same as in the first embodiment, a description thereof will not be repeated here.

On the other hand, in a case that the grasping forceps 30 is in the linear shape after the treatment of the target tissue P2, the determination result in step S16 is YES, and then the procedure goes to the above-described step S20. A description of the procedure of step S20 and thereafter will not be repeated here.

According to the medical manipulator 3 of the present embodiment with such a configuration, since the distal end portion of the grasping forceps 30 is moved closer to the proximal end side than the position at the time of receiving the operation instruction to change the angle, it is possible to suppress the grasping forceps 30 from contacting tissue or the like on the front side. That is, by moving the distal end portion of the grasping forceps 30 to the proximal end side without moving the distal end portion of the grasping forceps 30 to the distal end side, it is possible to achieve the above effects.

Further, by moving the distal end portion of the grasping forceps 30 linearly to the opening 11a of the channel 11, it is possible to shorten a moving path of the distal end portion of the grasping forceps 30 moves, and to reduce the movement time.

In the present embodiment, the endoscope control section 101 moves the distal end portion of the grasping forceps 30 linearly to the opening 11a of the channel 11 when the grasping forceps 30 is moved to the proximal end side, however, it is not indispensable. At least, it is sufficient that the distal end portion of the grasping forceps 30 can be moved closer to the proximal end side than the position at the time of receiving the operation instruction without moving to the distal end side.

### Fourth Embodiment

Next, a fourth embodiment of the present invention will be described with reference to FIGS. 2, 16 to 18, in which the same reference numerals are given to the same parts as in the embodiments, and a description thereof will not be repeated here.

As shown in FIG. 2, a medical manipulator 4 of the present embodiment includes an endoscope control section 111 instead of the endoscope control section 72 of the medical manipulator 1 in the first embodiment.

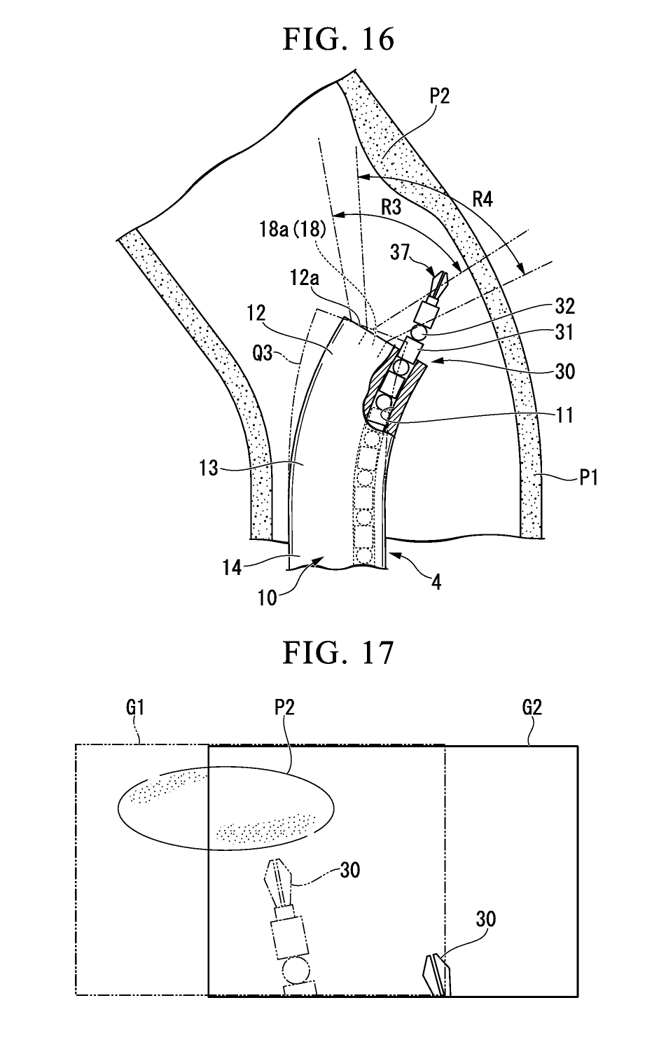

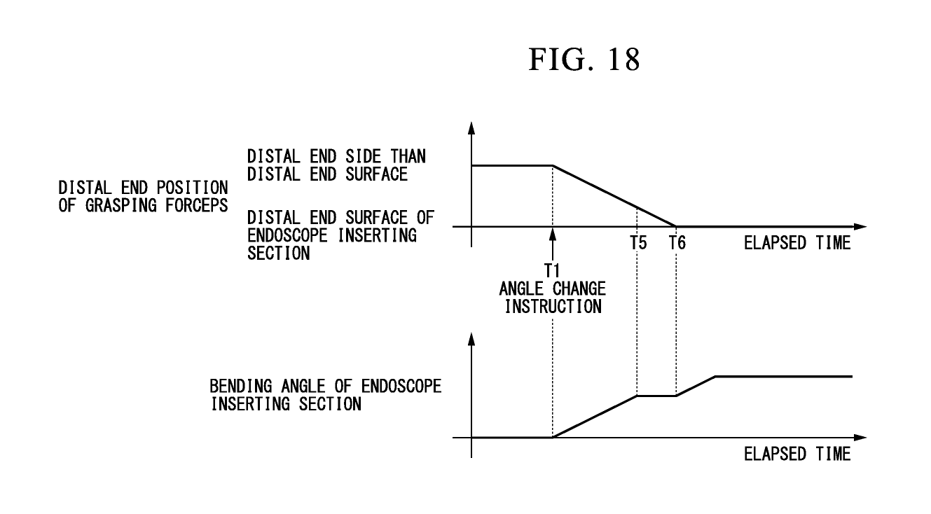

As shown in FIGS. 16 and 18, an arithmetic element of the endoscope control section 111 stores in a memory an initial viewing range R3 that is a viewing range of an imaging unit 18a with respect to a position Q3 of the endoscope inserting section 10 at the time of T1 when the operation instruction for bending the bending section 13 is received. As shown in FIG. 17, the endoscope control section 111 recognizes the boundary of the initial viewing range R3 by the shape or color of target tissue P2 or the like imaged into an image G1 in the initial viewing range R3. When the endoscope control section 111 receives the operation instruction, as shown in FIGS. 16 and 17, it is assumed that the distal end of the grasping forceps 30 is arranged in the viewing range of the imaging unit 18a. It is possible to determine whether the distal end of the grasping forceps 30 is in the initial viewing range R3 by performing a known image analysis for the view of the imaging unit 18a provided in the distal end surface 12a of the rigid distal end portion 12, the shape of the grasping forceps 30 protruding from the distal end surface 12a, and the image G1.

As shown in FIG. 18, the endoscope control section 111 simultaneously starts movement of the grasping forceps 30 to the proximal end side and bending of the bending section 13 of the endoscope inserting section 10, at the time of T1 when the operation instruction is received. Further, at the time of T5 when the distal end of the grasping forceps 30 reaches an edge part of the initial viewing range R3, the bending of the endoscope inserting section 10 is stopped, and the movement of the grasping forceps 30 to the proximal end side is performed. A viewing range R4 at the time of T5 is shown in FIG. 16. An image obtained by the imaging unit 18a at the time of T5 is as an image G2 shown in FIG. 17.

At the time of T6 shown in FIG. 18 when the entirety of the grasping forceps 30 is housed inside the channel 11 and the retraction operation of the grasping forceps 30 is finished, the bending of the endoscope inserting section 10 is restarted.

According to the medical manipulator 4 of the present embodiment with such a configuration, the bending section 13 of the endoscope inserting section 10 starts to be bent immediately after the operating section 50 generates the operation instruction to bend the bending section 13. Thus, when the operator O operates the operating section 50, it is possible to reduce discomfort of the operator O that the endoscope inserting section 10 is not likely to bend even though the operator O operates the operating section 50.

Since the bending of the endoscope inserting section 10 is started at the time of T1 when the operation instruction is received, the present embodiment may also be applied to a case where the bending section 13 is bent as the operator manually operates an angle knob or the like. In this case, a sensor used to detect that the angle knob is operated is provided in the vicinity of the angle knob, and the treatment section reciprocating motor 20 is provided. Further, at the same time when the sensor detects that the angle knob is operated, the grasping forceps 30 is moved to the proximal end side by the treatment section reciprocating motor 20.

### Fifth Embodiment

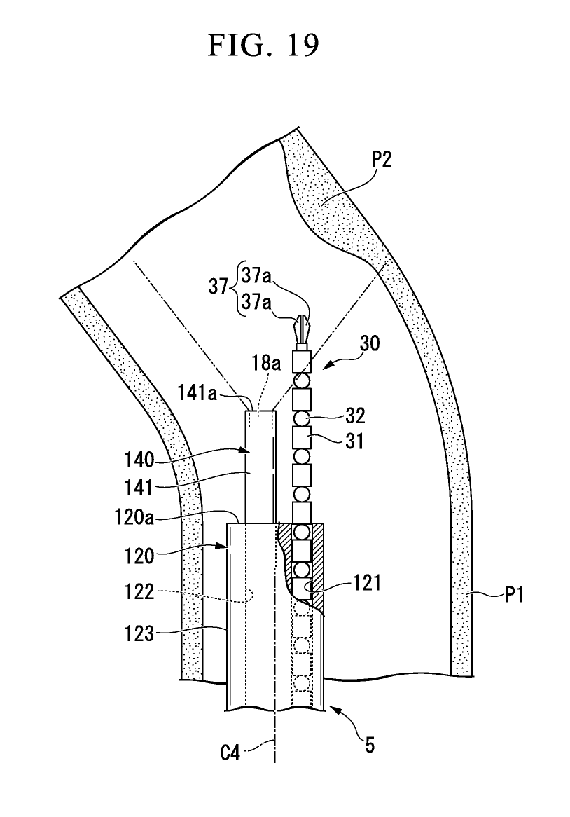

Next, a fifth embodiment of the present invention will be described with reference to FIGS. 19 to 22, in which the same reference numerals are given to the same parts as in the first embodiment, and the description thereof will not be repeated here.

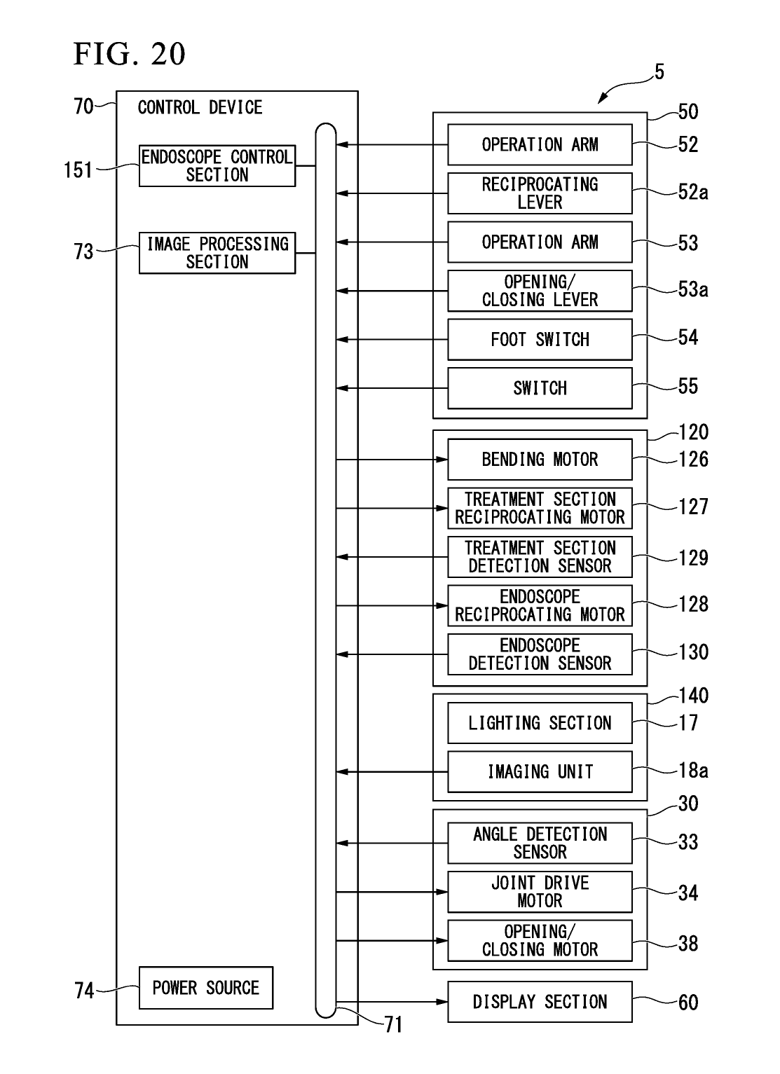

As shown in FIGS. 19 and 20, in a medical manipulator 5 of the present embodiment, grasping forceps 30 and an endoscope inserting section (imaging section) 140 are retractably inserted in lumens 121 and 122 formed in an overtube (inserting section) 120 being capable of moving advanced and retracted.

The lumens 121 and 122 are formed to extend in the direction of an axis line C4 of the overtube 120. On a distal end side of the overtube 120, a bending section 123 that has a configuration similar to the bending section 13 of the endoscope inserting section 10 is provided. A distal end portion of an operating wire (not shown) is connected to the bending section 123. By drawing a proximal end portion of the operating wire by a bending motor 126, it is possible to bend the bending section 123.

A treatment section reciprocating motor 127 and an endoscope reciprocating motor 128, and a treatment section detection sensor 129 and an endoscope detection sensor 130 having the same configurations as those of the treatment section reciprocating motor 20 and the treatment section detection sensor 21 of the first embodiment are respectively provided in the overtube 120.

It is possible to advance and retract the grasping forceps 30 inserted in the lumen 121 by the treatment section reciprocating motor 127, and to detect the position of the grasping forceps 30 in the direction of the axis line C4 by the treatment section detection sensor 129. Similarly, it is possible to advance and retract the endoscope inserting section 140 inserted in the lumen 122 by the endoscope reciprocating motor 128, and to detect the position of the endoscope inserting section 140 in the direction of the axis line C4 by the endoscope detection sensor 130.

The endoscope inserting section 140 is provided with the above-mentioned lighting section 17 (not shown in FIG. 19) and the imaging unit 18a in a distal end surface 141a of a rod-shaped main body (imaging section main body) 141 formed of a flexible material such as resin with the lighting section 17 and the imaging unit 18a being exposed outside. The endoscope inserting section 140 of the present embodiment does not include a bending section that is capable of bending by operation.

A switch 55 that switches an operating target by the operation arms 52 and 53 is provided on the operating section 50.

The switch 55 switches the target operated by the operation arms 52 and 53 between the endoscope inserting section 140, the grasping forceps 30 and the overtube 120 (hereinafter, referred to as “the endoscope inserting section 140 and the like”).

The control device 70 includes an endoscope control section 151 instead of the endoscope control section 72. The endoscope control section 151 is configured similarly to the endoscope control section 72, and controls the endoscope inserting section 140 and the like according to an operation instruction from the operating section 50.

A technique of using the medical manipulator 5 of the present embodiment with such a configuration will be described herein regarding a point that is different from the technique of using the medical manipulator 1 of the first embodiment. Hereinafter, it is assumed that the endoscope inserting section 140 protrudes closer to the front side than the distal end surface 120a of the overtube 120 after the treatment of the target tissue P2 and the grasping forceps 30 further protrudes to the front side than the endoscope inserting section 140. The grasping forceps 30 is in the linear shape.

The protrusion lengths of the grasping forceps 30 and the endoscope inserting section 140 from the distal end surface 120a of the overtube 120 are respectively detected by the treatment section detection sensor 129 and the endoscope detection sensor 130, and are outputted to the endoscope control section 151 through the bus 71.

The operator O can operate the overtube 120 by the operation arm 52 through operating the switch 55.

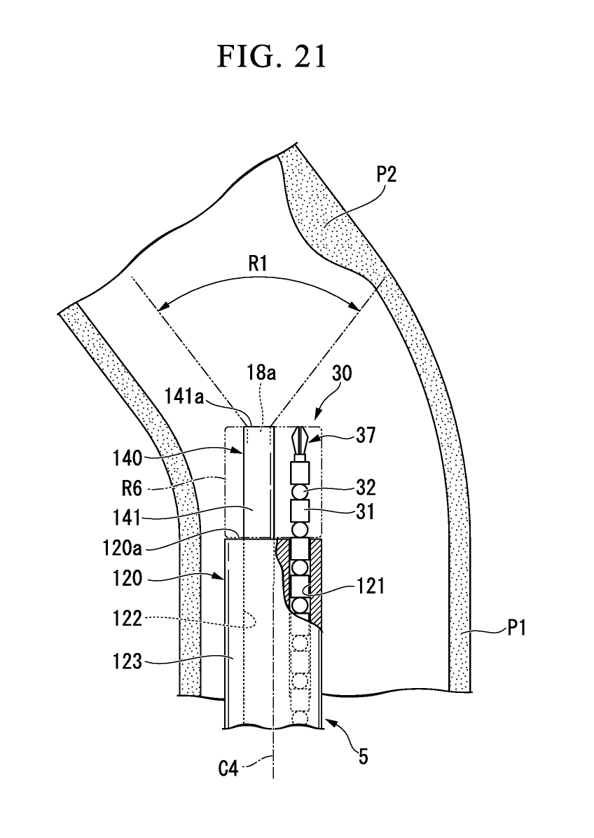

The endoscope control section 151 performs the retraction operation of the grasping forceps 30 in which the grasping forceps 30 is moved to the proximal end side until the distal end of the grasping forceps 30 reaches the position of the distal end surface 141a of the endoscope inserting section 140 in the direction of the axis line C4 as shown in FIG. 21 when the operation instruction to change the angle of the bending section 123 of the overtube 120 is received.

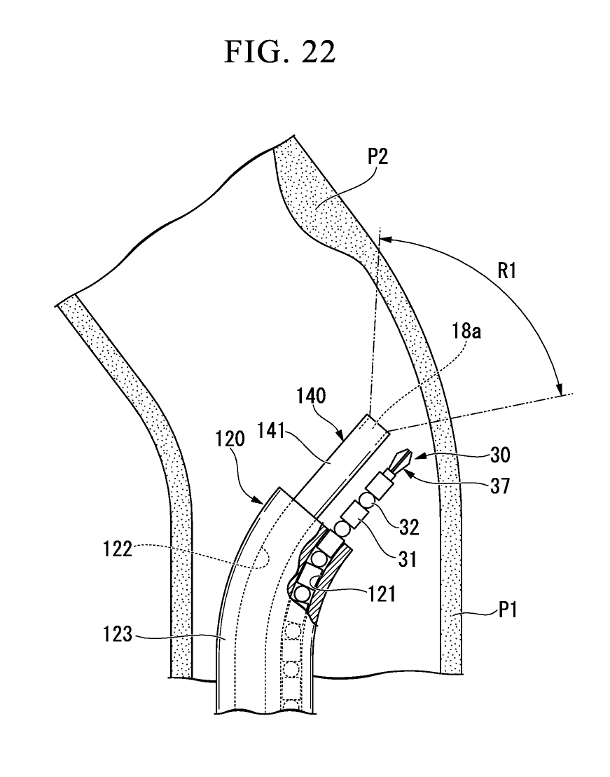

Further, after the retraction operation is finished, the bending section 123 of the overtube 120 is bent as shown in FIG. 22.

In general, it is considered that the grasping forceps 30 is hardly in contact with surrounding tissue even though the overtube 120 is bent in a state where the grasping forceps 30 is arranged in a region R6 (see FIG. 21) up to the distal end surface 141a of the endoscope inserting section 140 on the front side of the overtube 120.

According to the medical manipulator 5 of the present embodiment with such a configuration, when the overtube 120 is bent, it is possible to help prevent the grasping forceps 30 from contacting the surrounding tissue. Further, since the distal end of the grasping forceps 30 is moved toward the proximal end side only up to the position of the distal end surface 141a of the endoscope inserting section 140, it is possible to easily return the grasping forceps 30 in the viewing range R1 after bending the overtube 120.

In the present embodiment, the endoscope inserting section 140 is inserted in the lumen 122 of the overtube 120, however, the proximal end portion of the endoscope inserting section may be configured to be fixed to the distal end surface of the overtube 120.

Hereinbefore, the first to fifth embodiments of the present invention have been described with reference to the accompanying drawings, but specific configurations are not limited to these embodiments, and may include modifications or the like as long as they do not depart from the range within the gist of the present invention. Further, the combining of the configurations shown in the respective embodiments is possible as long as it is done appropriately.

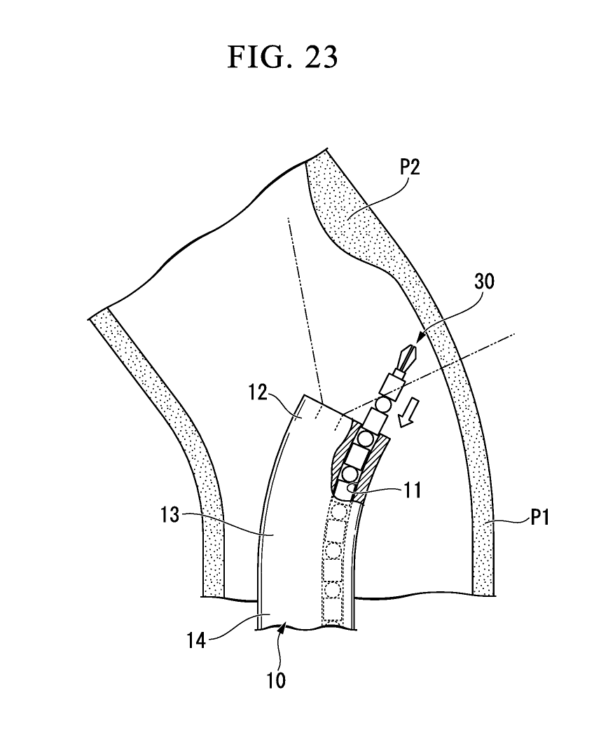

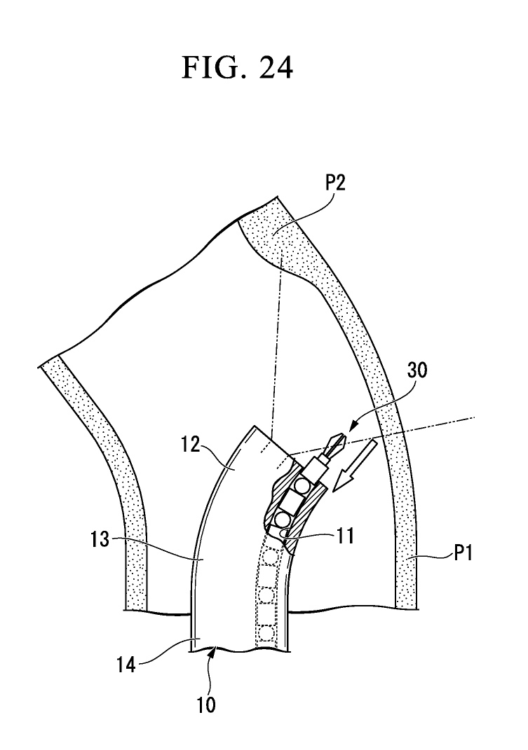

For example, in the first to fifth embodiments, when the operation arm 52 is operated, the amount of movement of the grasping forceps 30 toward the proximal end side is constant. However, as the amount of movement of the operation arm 52 operated by the operator O increases, the bending amount of the bending section indicated by the operation instruction for changing the angle of the bending section outputted from the operating section 50 may be controlled to be increased, and the amount of movement of the grasping forceps 30 toward the proximal end side may be controlled to be increased. Specifically, when the amount of movement of the operation arm 52 is slight, as shown in FIG. 23, the amount of movement of the grasping forceps 30 toward the proximal end side is reduced. On the other hand, when the amount of movement of the operation arm 52 is large, as shown in FIG. 24, the amount of movement of the grasping forceps 30 toward the proximal end side is increased.

With this configuration, it is possible to prevent the grasping forceps 30 from contacting the surrounding tissue adjusting to the movement amount of the operation arm 52. Further, since the movement amount of the grasping forceps 30 toward the proximal end side is changed according to the movement amount of the operation arm 52, it is possible to improve the operability of the operation arm 52. When the movement amount of the operation arm 52 is slight, it is possible to reduce the time necessary to return the grasping forceps 30 to the distal end side after changing the angle of the bending section.

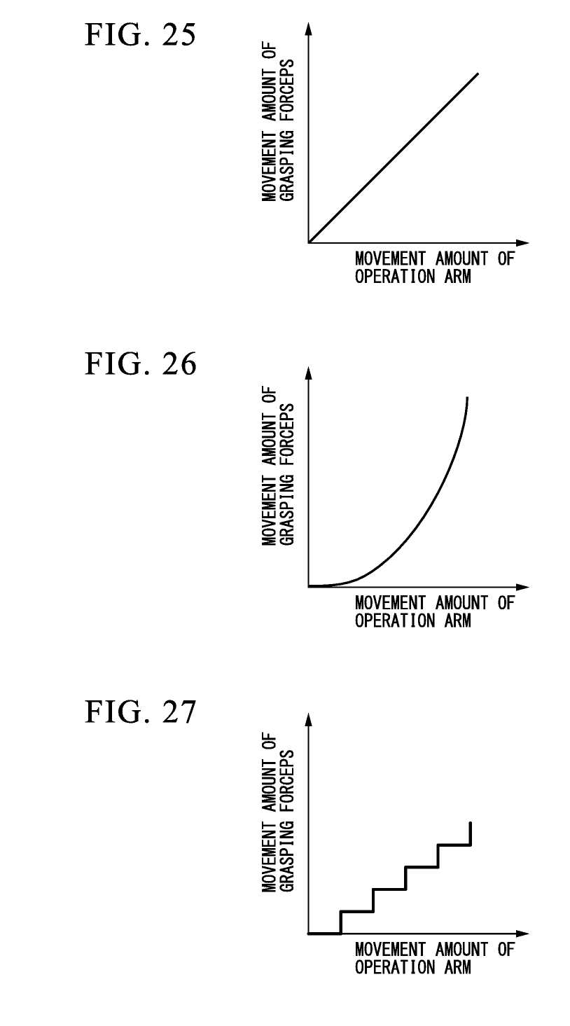

The relationship between the amount of movement of the operation arm 52 and the amount of movement of the grasping forceps 30 toward the proximal end side may be proportional as shown in FIG. 25. Further, as indicated by a quadratic curve shown in FIG. 26, the relationship may be a curved relationship or a discrete (staged) relationship is as shown in FIG. 27 which is not proportional may be used.

The amount of movement of the grasping forceps 30 toward the proximal end side may be associated to the speed or acceleration of the operation arm 52 instead of the amount of movement of the operation arm 52.

In the first to fourth embodiments, the grasping forceps 30 is inserted in the channel 11 of the endoscope inserting section 10, however, the proximal end portion of the grasping forceps may be configured to be fixed to the distal end surface 12a of the endoscope inserting section 10. In this case, by bending the grasping forceps, the distal end side of the grasping forceps moves to the proximal end side.

The treatment section is the grasping forceps 30, however, the type of the treatment section is not limited thereto. As the treatment section, a high-frequency treatment instrument such as a snare or a knife, an injector, a needle holder or the like may be appropriately used, instead of the grasping forceps 30.

The endoscope inserting section 10 is a flexible inserting section having the flexible tube part 14. However, the endoscope inserting section may be a so-called rigid inserting section having no flexibility. In a case that the endoscope inserting section is a rigid inserting section, the endoscope inserting section inserted in an opening (single port) formed in a body wall of a patient swings around the opening, the medical manipulator of the present invention may be used effectively.

A part of the grasping forceps 30 is a rigid treatment section, however, a flexible treatment section having no rigid part over the entire length in the longitudinal direction may be used. In this case, even though the treatment section is bent before the treatment section is moved to the proximal end side, the treatment section is moved to the proximal end side without deforming the treatment section into the linear shape.

The angle detection sensor 33 and the joint drive motor 34 are provided in the joint part 32 of the grasping forceps 30. However, for example, the joint drive motor 34 may be provided on the proximal end side with reference to the tubular body 31 and the joint part 32 in the grasping forceps 30, and the joint part 32 may be rotated by a wire (not shown) or the like connected to a drive shaft of the joint drive motor 34. The number of rotations of the drive shaft of the joint drive motor 34 provided on the proximal end side may be detected by the angle detection sensor 33 such as an encoder, and an angle formed by the adjacent tubular bodies 31 may be calculated from the detected number of rotations. In this case, the angle detection sensor 33 is provided on a portion other than the joint part 32.

In the first to fourth embodiments, the endoscope inserting section 10 includes the lighting section 17 or the imaging section 18, however, an inserting section that does not include these sections may also be used in the present invention. In this case, the treatment is performed while observing a distal end portion of the inserting section by introducing a known endoscope into the body. This is also applied to the fifth embodiment.

The imaging unit 18a is a CCD, however, the imaging unit is not limited thereto, and for example, it is appropriate to use a CMOS, an image fiber or the like.

The endoscope control section of the control device 70 may have only the retraction mode as the control mode.

Further, the present invention is not limited to the above description, and is only defined by the scope of the appended claims.

In the medical manipulator and the control method of the medical manipulator in the above-described embodiments, the endoscope control section moves the grasping forceps to the proximal end side when the operation instruction to bend the endoscope inserting section is received. Thus, the swiveling range of the grasping forceps becomes narrowed when the distal end side of the endoscope inserting section is bent. Accordingly, it is possible to suppress the grasping forceps from contacting the wall of the surrounding tissue or the like.

What is claimed is:

1. A medical manipulator comprising:
an inserting section that is configured to be inserted in a body;
a treatment section which is provided on a distal end portion of the inserting section;
an operating section that operates the inserting section; and
a control section that includes a retraction mode in which the treatment section is retracted to a proximal end side when the control section receives an instruction to move the inserting section from the operating section.

2. The medical manipulator according to claim 1, wherein
the control section moves the treatment section to the proximal end side only when the control section receives the instruction to move the inserting section in a direction intersecting an axis line of the inserting section from the operating section.

3. The medical manipulator according to claim 1, wherein
when the treatment section is moved to the proximal end side, the control section moves a distal end portion of the treatment section closer to the proximal end side than a position of the distal end portion of the treatment section when the control section receives the instruction.

4. The medical manipulator according to claim 3, wherein
the control section stores a position of an arrangement reference line of the treatment section defined along a longitudinal direction of the treatment section when the control section receives the instruction, and
the control section moves the treatment section to the proximal end side while maintaining a state where the treatment section is arranged on the arrangement reference line.

5. The medical manipulator according to claim 1, wherein
the control section deforms the treatment section into a linear shape extending to a distal end side thereof when the control section receives the instruction before the treatment section is moved to the proximal end side.

6. The medical manipulator according to claim 3, wherein
a channel which has an opening in a distal end surface of the inserting section is formed in the inserting section,
the treatment section is inserted in the channel being capable of moving advanced and retracted, and
the control section moves the treatment section so as to be housed an entirety of the treatment section inside the channel when the control section receives the instruction.

7. The medical manipulator according to claim 1, wherein
the control section moves the inserting section after finishing a retraction operation of the treatment section.

8. The medical manipulator according to claim 1, wherein
the control section moves the inserting section after receiving the instruction and before finishing a retraction operation of the treatment section.

9. The medical manipulator according to claim 1, wherein
the control section performs control so that an amount of a movement of the treatment section toward the proximal end side is increased as an amount of a movement of the inserting section indicated by the instruction is increased.

10. The medical manipulator according to claim 1, wherein
the control section has a non-retraction mode in which the treatment section is not moved to the proximal end side even though receiving the instruction to move the inserting section from the operating section, and
the control section is capable of switching the retraction mode and the non-retraction mode.

11. The medical manipulator according to claim 1, wherein
an imaging section which has an imaging unit capable of obtaining an image of a subject in a visual field range is provided on the distal end portion of the inserting section,
a distal end of the treatment section is arranged in the visual field range of the imaging unit when the control section receives the instruction,
the control section stores an initial visual field range that is the visual field range of the imaging unit when the control section receives the instruction and starts a movement of the inserting section, and
the movement of the inserting section is stopped when the distal end of the treatment section reaches an edge part of the initial viewing range.

12. The medical manipulator according to claim 7, further comprising:
an imaging section that includes an imaging section main body that is provided to protrude from a distal end surface of the inserting section and an imaging unit that is provided in a distal end portion of the imaging section main body and that is capable of obtaining an image of a subject, wherein
the control section stops the retraction operation of the treatment section when the treatment section moves to the proximal end side so that a distal end of the treatment section reaches the position of a distal end of the imaging section in a direction of an axis line of the inserting section.

13. A medical manipulator control method of controlling a medical manipulator that includes an inserting section that is configured to be inserted in a body, a treatment section that is provided in a distal end portion of the inserting section, and an operating section that operates the inserting section, the method comprising:
a process of moving the treatment section to a proximal end side when the control section receives an instruction to move the inserting section from the operating section.

14. The medical manipulator control method according to claim 13, wherein the treatment section is moved to the proximal end side only when the instruction to move the inserting section in a direction intersecting an axis line of the inserting section is received from the operating section.
